(19) United States
(12) Patent Application Publication
(10) Pub. No.: US 2024/0331835 A1
(43) Pub. Date: Oct. 3, 2024

(54) SYSTEM AND METHODS FOR AUTOMATED PERMISSIONING OF MEDICAL PROCEDURES AND TESTS

(71) Applicant: Optum, Inc., Minnetonka, MN (US)

(72) Inventors: Michael Nicholas Peresie, Milton, GA (US); Srilekha Akula, Fremont, CA (US)

(21) Appl. No.: 18/193,752

(22) Filed: Mar. 31, 2023

(51) Int. Cl. G16H 20/40; G16H 10/60; G16H 40/67

(52) U.S. Cl. CPC G16H 20/40; G16H 10/60; G16H 40/67

(57) ABSTRACT

A system and methods for automated permissioning of medical procedures or tests, and optional health care claim generation, are disclosed. The method includes obtaining an electronic medical record for a patient, determining a clinical appropriateness of the medical procedure or test from the electronic medical record, generating a preapproval decision for the medical procedure or test, presenting the preapproval decision to the health care provider, generating a summary of how the clinical appropriateness score was determined, and transmitting the summary of the evaluation to a remote computing device.

### BACKGROUND

In the U.S. health care system, health care providers are generally required to submit authorization requests to health care payors prior to initiating and/or performing medical procedures and tests. Health care payors are then responsible for processing these requests—often manually—and returning, to the heath care provider, either a preapproval or denial of the request. Not only does this place undue burden on health care payors, but traditional permissioning processes often incur significant delays in treatment and billing while providers wait for requests to be reviewed and approved. These delays can carry over to the health care claims adjudication and settlement processes, as health care payors often also need to manually review received health care claims and patient files. In some cases, health care claims adjudication can take upwards of thirty days. Missing or incomplete documentation can result in even further delays and inefficient communications between providers and payors.

### SUMMARY

One implementation of the present disclosure is a system including: a processor; and memory having instructions stored thereon that, when executed by the processor, cause the system to: obtain an electronic medical record for a patient, wherein the electronic medical record includes a recommendation from a health care provider for a medical procedure or test and a medical history for the patient stored as both structured and unstructured data; determine a clinical appropriateness of the medical procedure or test by: i) extracting a subset of both the structured and unstructured data from the electronic medical record relevant to the medical procedure or test, and ii) generating a score indicative of the clinical appropriateness based on the subset of both the structured and unstructured data; generate a preapproval decision for the medical procedure or test based on the score, wherein the medical procedure or test is preapproved if the score meets or exceeds a threshold value; present the preapproval decision to the health care provider; generate a summary of the preapproval decision, wherein the summary includes an indication of the preapproval decision and an indication of clinical appropriateness criteria that impacted the preapproval decision; and transmit the summary of the evaluation to a remote computing device.

In some implementations, the unstructured data included in the electronic medical record includes text-based notes entered by the health care provider, and extracting the subset of both the structured and unstructured data includes performing keyword extraction on the text-based notes using a natural language processing model.

In some implementations, the score indicative of the clinical appropriateness is generated using a classification model, where the classification model is a machine learning model, and wherein the subset of both the structured and unstructured data are provided as inputs to the classification model.

In some implementations, the score indicative of the clinical appropriateness is generated using a rules-based model, wherein the rules-based model compares data points of the subset of both the structured and unstructured data to a set of clinical appropriateness criteria.

In some implementations, the instructions further cause the system to: identify procedure and diagnostic codes from the electronic medical record using an artificial intelligence model; generate an electronic health care claim based on the procedure and diagnostic codes; and transmit the electronic health care claim to the remote computing device.

In some implementations, the instructions further cause the system to: receive payment for the electronic health care claim from the remote computing device; and process the payment to preliminarily settle the electronic health care claim.

In some implementations, to obtain the electronic medical record for the patient, the instructions further cause the system to: receive a request for preapproval of the medical procedure or test from the health care provider; and retrieve the electronic medical record from a database.

In some implementations, presenting the preapproval decision to the health care provider includes at least one of: i) transmitting the preapproval decision to a second remote computing device, or ii) displaying the preapproval decision on a user interface.

In some implementations, the remote computing device is a first computer associated with a health care payor, and wherein the electronic medical record is received from a second computer associated with the health care provider.

Another implementation of the present disclosure is a computer-implemented method including: obtaining an electronic medical record for a patient, wherein the electronic medical record includes a recommendation from a health care provider for a medical procedure or test and a medical history for the patient stored as both structured and unstructured data; determining a clinical appropriateness of the medical procedure or test by: i) extracting a subset of both the structured and unstructured data from the electronic medical record relevant to the medical procedure or test, and ii) generating a score indicative of the clinical appropriateness based on the subset of both the structured and unstructured data; generating a preapproval decision for the medical procedure or test based on the score, wherein the medical procedure or test is preapproved if the score meets or exceeds a threshold value; presenting the preapproval decision to the health care provider; generating a summary of the preapproval decision, wherein the summary includes an indication of the preapproval decision and an indication of clinical appropriateness criteria that impacted the preapproval decision; and transmitting the summary of the evaluation to a remote computing device.

In some implementations, the unstructured data included in the electronic medical record includes text-based notes entered by the health care provider, and wherein extracting the subset of both the structured and unstructured data includes performing keyword extraction on the text-based notes using a natural language processing model.

In some implementations, the score indicative of the clinical appropriateness is generated using a classification model, wherein the classification model is a machine learning model, and wherein the subset of both the structured and unstructured data are provided as inputs to the classification model.

In some implementations, the score indicative of the clinical appropriateness is generated using a rules-based model, wherein the rules-based model compares data points of the subset of both the structured and unstructured data to a set of clinical appropriateness criteria.

In some implementations, the computer-implemented method further includes: identifying procedure and diagnostic codes from the electronic medical record using an artificial intelligence model; generating an electronic health care claim based on the procedure and diagnostic codes; and transmitting the electronic health care claim to the remote computing device.

In some implementations, the computer-implemented method further includes: receiving payment for the electronic health care claim from the remote computing device; and processing the payment to preliminarily settle the electronic health care claim.

In some implementations, obtaining the electronic medical record for the patient includes: receiving a request for preapproval of the medical procedure or test from the health care provider; and retrieving the electronic medical record from a database.

In some implementations, presenting the preapproval decision to the health care provider includes at least one of: i) transmitting the preapproval decision to a second remote computing device, or ii) displaying the preapproval decision on a user interface.

In some implementations, the summary further includes a copy of the electronic medical record or a portion of the electronic medical record.

In some implementations, the electronic medical record is obtained upon detecting that the electronic medical record has been created or updated by the health care provider.

Yet another implementation of the present disclosure is a non-transitory computer readable medium having instructions stored thereon that, when executed by a processor, cause a device to: obtain an electronic medical record for a patient, wherein the electronic medical record includes a recommendation from a health care provider for a medical procedure or test and a medical history for the patient stored as both structured and unstructured data; determine a clinical appropriateness of the medical procedure or test by: i) extracting a subset of both the structured and unstructured data from the electronic medical record relevant to the medical procedure or test, and ii) generating a score indicative of the clinical appropriateness based on the subset of both the structured and unstructured data; generate a preapproval decision for the medical procedure or test based on the score, wherein the medical procedure or test is preapproved if the score meets or exceeds a threshold value; present the preapproval decision to the health care provider; generate a summary of the preapproval decision, wherein the summary includes an indication of the preapproval decision and an indication of clinical appropriateness criteria that impacted the preapproval decision; and transmit the summary of the evaluation to a remote computing device.

Additional features will be set forth in part in the description which follows or may be learned by practice. The various features described herein will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1 is a block diagram of an example medical procedure/test permissioning and health care claims adjudication architecture, according to some implementations.

FIG. 2 is a block diagram of a permissioning tool as included in the architecture of FIG. 1, according to some implementations.

FIG. 3 is a flow chart of a process for automated preapproval of medical procedures and tests, according to some implementations.

Various objects, aspects, and features of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a system and methods for automated permissioning of medical procedures and tests are shown, according to various implementations. The term “permissioning,” as used throughout the present disclosure, generally refers to a process of authorizing a health care provider (e.g., a physician) to initiate and/or perform a medical procedure/test with respect to a patient's health insurance or other health care payor. Generally, health care providers request authorization for medical procedures and tests from health care payors (e.g., health insurance companies) prior to performing said procedures and/or tests; hence, “permissioning” can, more specifically, refer to a process for preapproval and/or preauthorization or medical procedures and tests. In addition, the system and methods described herein can facilitate the automatic generation and/or adjudication of health care claims, in various implementations.

As mentioned above, requests for medical procedures and tests are traditionally manually reviewed by health care payors on a case-by-case basis, which introduces significant delays in treatment and claims adjudication. For example, a request to initiate a medical procedure can take days to be preapproved which can cause delays in patient care and/or billing. In cases where time is of the essence, health care providers may be forced to proceed with medical procedures and tests without waiting for preapproval, which can result in delays in adjudicating the associated health care claims and even increased costs for patients (e.g., if a procedure or test is not covered or ends up being denied). Existing manual review processes are also prone to human error and discrepancies between reviewers. In some cases, health care payors end up reviewing a patient's medical records multiple times for a single medical procedure or test (e.g., for preapproval of the procedure/test, when adjudicating a claim, etc.), which is inefficient and places additional burden on health care payors and their systems.

The disclosed system and methods can address these and other limitations of existing health care preapproval and adjudication processes by automatically determining a clinical appropriateness and/or necessity of a medical procedure or test based on a patient's electronic medical record. For example, the patient's medical record may be evaluated to generate a score indicative of clinical appropriateness/necessity and, if the score meets or exceeds a threshold value, the medical procedure/test is preapproved without manual intervention. In this manner, preapproval of medical procedures and tests can be almost instantaneously returned to health care providers to reduce or eliminate wait times, while alleviating at least part of the burden on health care payors. Notably, health care professionals can proceed with performing procedures and tests without seeking prior approval from payors, assuming that a recommend medical procedure or test meets certain preapproval criteria. In addition, an electronic health care claim can be automatically generated for procedures/tests that are preapproved. In some implementations, an automatically generated electronic health care claim can be preliminarily settled with a health care payor to further reduce overall adjudication and settlement times. In at least one configuration, the disclosed system and methods are implemented as a “permissioning tool,” which is described in greater detail below.

### Automated Permissioning System

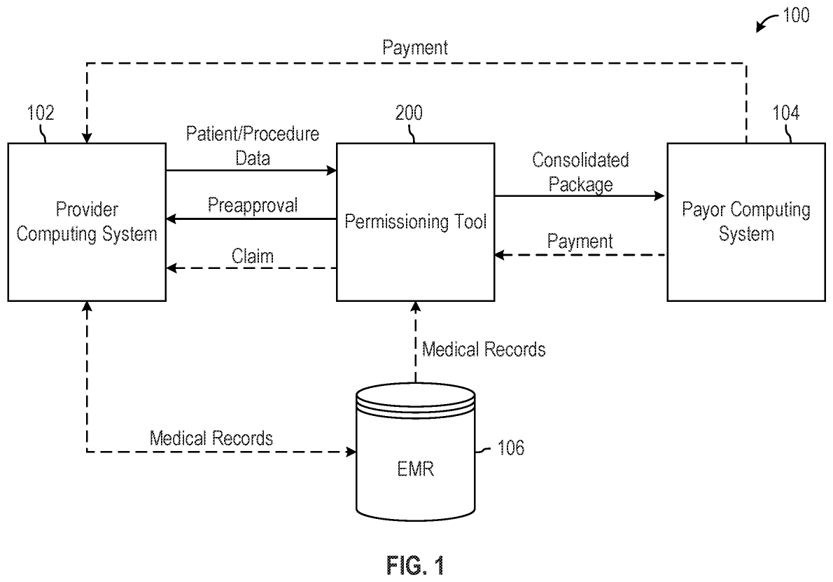

Referring now to FIG. 1, a block diagram of an example medical procedure/test permissioning and health care claims adjudication architecture 100 is shown, according to some implementations. At a high level, architecture 100 generally illustrates communications between multiple computing devices for the preapproval of a medical procedure or test, and optional resulting health care claims generation. Architecture 100 is generally shown to include a provider computing system 102, a payor computing system 104, and a Permissioning Tool 200. Optionally, architecture 100 may include a separate electronic medical records (EMR) database 106; however, as described below, EMR database 106 can also be incorporated into one or more of provider computing system 102, payor computing system 104, or Permissioning Tool 200. It should be appreciated that the specific configuration of architecture 100 shown in FIG. 1 is not intended to be limiting; rather, the various components of architecture 100 and their corresponding functionalities may be implemented in a variety of different ways.

Provider computing system 102 is generally any computing system or suitable computing device (e.g., a workstation, a server, etc.) that is operated by a health care provider or other user that can input/update electronic medical records for a patient. For the sake of simplicity, provider computing system 102 is generally described herein as a computer operated by a health care provider (e.g., a physician). As an example, provider computing system 102 may be a workstation (e.g., a computer device coupled to a server), a desktop computer, a laptop, or the like operated by a physician. Provider computing system 102 therefore generally includes at least one processing circuit having at least one processor for executing instructions (e.g., computer code) stored on memory, to implement various functions described herein. In many cases, provider computing system 102 also includes a user interface that can display information (e.g., pictures, text, graphics, etc.) to a user and that can receive user inputs. For example, provider computing system 102 can include a display screen, a keyboard, and/or a mouse.

Along similar lines, payor computing system 104 is generally any computing system or suitable computing device (e.g., a workstation, a server, etc.) that is operated by an entity that submits payment for electronic health care claims. For example, provider computing system 104 may be a computer operated by a health care payor, such as an insurance company. Payor computing system 104 generally includes at least one processing circuit having at least one processor for executing instructions (e.g., computer code) stored on memory, to implement various functions described herein. In some implementations, payor computing system 104 can include a user interface that can display information (e.g., pictures, text, graphics, etc.) to a user and that can receive user inputs. For example, payor computing system 104 can include a display screen, a keyboard, and/or a mouse, similar to provider computing system 102.

Permissioning Tool 200, as briefly mentioned above, is generally configured to generate a preapproval decision for medical procedures or tests based, at least in part, on a patient's electronic medical records and other data obtained from a health care provider (e.g., that operates provider computing system 102). Permissioning Tool 200 may also be configured to auto-code and generate electronic health care claims, as discussed in greater detail below. Permissioning Tool 200 is generally implemented via any suitable computing device. In some implementations, Permissioning Tool 200 is hosted on and/or includes its own (e.g., stand-alone) computing device, such as a server or desktop computer. In other implementations, Permissioning Tool 200 is hosted on a shared computing device or a general-purpose computer. For example, Permissioning Tool 200 may be hosted on a server that performs other functions. In some implementations, Permissioning Tool 200 is hosted on an intermediary computing system, such as a computer operated by a clearinghouse, which facilitates communications between provider computing system 102 and payor computing system 104. In some implementations, Permissioning Tool 200 is hosted on a cloud server. In some such implementations, both provider computing system 102 and payor computing system 104 may communicate with Permissioning Tool 200 remotely (e.g., via the Internet). In yet other implementations, Permissioning Tool 200 may be hosted on either provider computing system 102 or payor computing system 104

It should be understood that, while only a single one of provider computing system 102 and payor computing system 104 are shown in the example of FIG. 1, architecture 100 may include any number of provider and/or payor computing systems. Likewise, architecture 100 may include multiple of EMR database 106. For example, multiple different health care providers and/or payors may utilize Permissioning Tool 200 to improve their preauthorization and/or claims adjudication processes; thus, as generally described herein, Permissioning Tool 200 may be generally configured to communicate with multiple different providers and payors. While not illustrated, it should also be appreciated that provider computing system 102, payor computing system 104, EMR database 106, and/or Permissioning Tool 200 may communicate via any suitable wired or wireless communication means. In this regard, the components of architecture 100 can communicate via direct connections or by any suitable network. For example, in some implementations, provider computing system 102, payor computing system 104, and payor computing system 104 are connected via the Internet using either wired or wireless connections.

EMR database 106 is generally a database of electronic medical records for one or more persons. In some implementations, EMR database 106 maintains electronic medical records for multiple patients of a particular health care provider. In some implementations, EMR database 106 maintains electronic medical records for multiple patients of multiple different health care providers. As shown in FIG. 1, in some implementations, EMR database 106 is separate from any of provider computing system 102, payor computing system 104, or Permissioning Tool 200. In some such implementations, EMR database 106 can be, or can be hosted by, a separate computing device (e.g., a server). For example, EMR database 106 may be hosted on a dedicated server or a secure cloud server that is accessible by provider computing system 102. In other implementations, EMR database 106 may be hosted and/or maintained by provider computing system 102 or Permissioning Tool 200. In yet other implementations, EMR database 106 can be hosted by payor computing system 104. In any case, at least provider computing system 102 and Permissioning Tool 200 may be in communication with EMR database 106, such that provider computing system 102 can input and/or update electronic medical records and Permissioning Tool 200 can reference/retrieve electronic medical records.

As described herein, an “electronic medical record”—or EMR—is an electronic file or record that contains notes and information collected by one or more medical professional relating to a patient, typically for use in diagnosis and/or treatment. In other words, an EMR is a digital version of the paper charts that were traditionally used in health care facilities to track patient information. EMRs generally include a partial medical history for a patient. It should be appreciated that “electronic medical records,” as described herein, may also refer to electronic health records (EHRs) which, while different from EMRs, generally contain similar information. Generally, EMRs contain a variety of health information for a patient, including, but not limited to, the patient's medical history, diagnoses, medications, treatment plans, immunization dates, allergies, radiology images, and laboratory and test results. In some cases, an EMR can include biometric and/or demographic information for a user (e.g., height, weight, age, gender, blood pressure, heart rate, etc.).

Often, EMRs also include notes entered by one or more medical professionals. For example, an EMR may include one or more text-based entries that detail a physician's observations, recommendations, and other notes relating to the patient. In some implementations, an EMR contains both structured and unstructured data. As will be appreciated by those of ordinary skill in the art, structured data generally refers to quantitative data that may follow a predefined format. Structured data often includes data in the form of numbers and values. Examples of structured data in health care include a patient's biometric and/or demographic information. Unstructured data is generally qualitative data that does not necessarily follow a predefined format. Examples of unstructured data in health care include the aforementioned notes entered by medical professionals (e.g., free-text), medical images, video or audio files, and the like. It should be appreciated that, in some cases, EMRs can also or alternatively include semi-structured data.

### Example Use-Case

To better understand architecture 100, consider the following example use-case where a health care provider sees a patient for a particular ailment, such as chronic migraines or a broken bone due to an accident. Generally, during or after a visit with the patient, the health care provider will create or update the patient's electronic medical record to include the patient's biometric information, demographic information, and/or other notes about the visit. For example, the health care provider may enter observations about the patient's condition, patient answers to diagnostic questions, and the like. In some cases, the health care provider also enters a recommendation for a diagnostic test or medical procedure. For example, the health care provider may recommend medical imaging (e.g., computed tomography (CT) or magnetic resonance imaging (MRI)) for a patient with chronic migraines. For a patient with a broken arm, the health care provider may recommend surgery to set the bones. In any case, the health care provider may enter these recommendations as notes (e.g., free-text) in the patient's EMR.

To begin the automated permissioning process, as described herein, Permissioning Tool 200 may obtain the recommendation for a medical procedure or test along with the patient's EMR. Alternatively, Permissioning Tool 200 may retrieve/reference the patient's EMR responsive to receiving/obtaining the recommendation for a medical procedure or test. In some implementations, the patient's EMR and/or the recommendation for a medical procedure or test may be sent directly to, or entered into, Permissioning Tool 200. For example, Permissioning Tool 200 may be accessible to the health care provider through a web page, software application, or other interface. In some implementations, Permissioning Tool 200 can detect that the patient's EMR has been created or updated, such as by monitoring EMR database 106, and may automatically determine whether the EMR contains a recommendation for a procedure or test. As will be described in greater detail below, Permissioning Tool 200 may be configured to evaluate both the structured and unstructured data in the patient's EMR to determine whether the EMR includes a recommendation for a procedure/test. In some implementations, Permissioning Tool 200 can, itself, be configured to generate recommendations for procedures or tests based on the information in the patient's EMR.

After identifying (or generating) a recommendation for a procedure or test, Permissioning Tool 200 may evaluate the patient's EMR to determine a medical necessity and/or clinical appropriateness-herein jointly referred to as “clinical appropriateness”—of the procedure or test. In some implementations, Permissioning Tool 200 uses a rules-based model or a classification model (e.g., an artificial intelligence (AI) model) to determine clinical appropriateness. In some such implementations, the model is trained on or references a data set of clinical appropriateness parameters/criteria. In some implementations, the model is trained using medical guidelines (e.g., provided by a regulatory body) and/or historical health care claims data, as discussed in greater detail below. In some implementations, Permissioning Tool 200 extracts relevant data points or features from the patient's EMR, based on the recommended procedure or test, and provides the extracted data points or features as inputs to said model. The model may then output a score indicative of clinic appropriateness. In some implementations, the score is a prediction of whether the procedure/test is clinically appropriate. For example, in implementations where the model is a classification model (e.g., an AI model), the output may be a classification of “approved” or “denied,” along with a confidence score in the classification. In other implementations, the model can simply output a determination of whether the procedure/test is preapproved or denied based on the patient's EMR.

Subsequently, Permissioning Tool 200 can return an indication of the preapproval decision to provider computing system 102. In some implementations, the indication is displayed to a user of provider computing system 102 (e.g., the health care provider). For example, the preapproval decision may be displayed on a screen of provider computing system 102 and/or may be indicated in the patient's EMR. Notably, Permissioning Tool 200 can generate a preapproval decision very quickly—in less than a second, in some cases-providing a near instant response to health care providers. In some implementations, if a medical procedure or test is deemed clinically appropriate, and therefore preapproved, Permissioning Tool 200 is also configured to automatically generate an electronic health care claim based on the procedure/test. In particular, as part of or in addition to evaluating the patient's EMR. Permissioning Tool 200 can automatically identify diagnosis, procedure, and/or other billing codes, which are used to populate an electronic health care claim. As discussed in greater detail below, Permissioning Tool 200 may utilize an AI model, such as a natural language processing (NLP) model, to identify diagnosis, procedure, and/or other billing codes from the unstructured data on the patient's EMR.

The auto-generated electronic health care claim can then be shared with payor computing system 104 to initiate an adjudication process. Additionally, or alternatively, the electronic health care claim can be shared with provider computing system 102. In some implementations, the electronic health care claim is included in a consolidated data package that is transmitted to payor computing system 104 based on the clinical appropriateness/preapproval evaluation discussed above. Specifically, the consolidated data package may include the electronic health care claim and an indication of the preapproval decision. In some implementations, the consolidated data package further includes a summary of how the preapproval decision was determined. In some such implementations, the summary indicates preapproval or “clinical appropriateness” criteria that impacted the preapproval decision-either positively or negatively. For example, the summary may indicate various data points that were considered, the rules or parameters that were met by the patient's EMR data, and any other information that was influential in approving or denying the medical procedure/test. In this manner, the health care payor (e.g., that operates payor computing system 104) is provided with transparency into the preapproval decision. In some implementations, the consolidated data package further includes a copy of at least a portion of the patient's EMR and/or provides payor computing system 104 with a link for access to the patient's EMR.

In some implementations, Permissioning Tool 200 is further and/or optionally configured to preliminarily settle the auto-generated electronic health care claim with payor computing system 104 based on contract terms with the health care payor. More specifically, payor computing system 104 may adjudicate the electronic health care claim, such as by performing a spot auditing based on the data in the consolidated data package. Payor computing system 104 may then submit payment to Permissioning Tool 200 and/or provider computing system 102. In some implementations, payment is received and optionally held by Permissioning Tool 200 as a preliminary settlement (e.g., until a final electronic health care claim or final payment is requested by provider computing system 102).

### Permissioning Tool

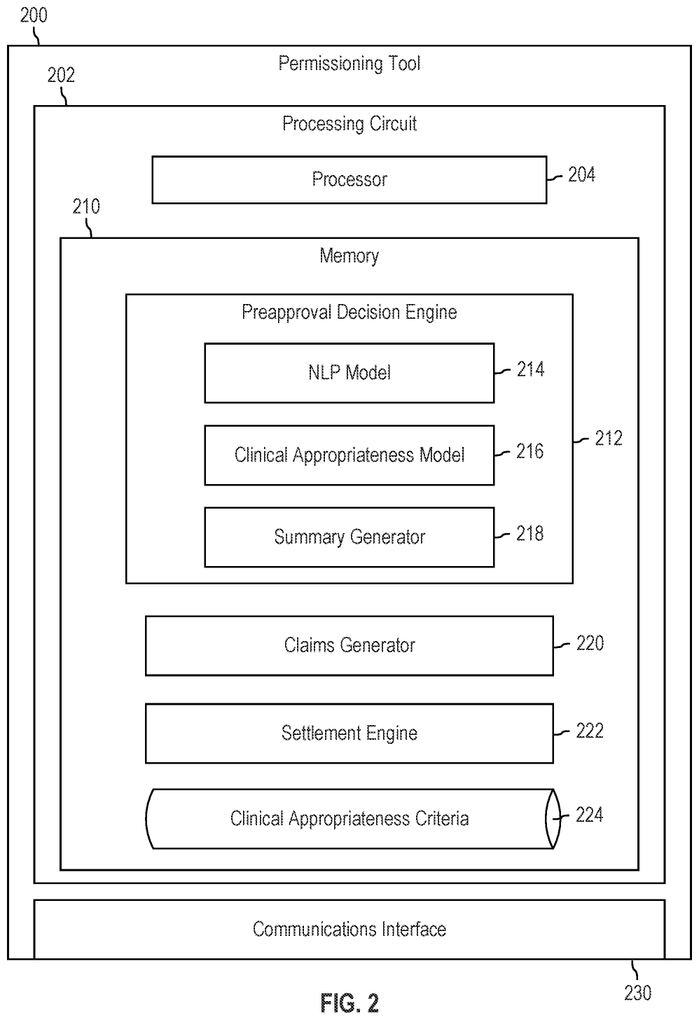

Referring now to FIG. 2, a detailed block diagram of Permissioning Tool 200 is shown, according to some implementations. While not explicitly referenced herein, it will be appreciated that the following description of Permissioning Tool 200 builds on, and may be better understood with reference to, the preceding description of FIG. 1. Permissioning Tool 200 is generally shown to include a processing circuit 202 that includes a processor 204 and a memory 210. Processor 204 can be a general-purpose processor, an applicant specific integrated circuit (ASIC), one or more field programmable gate arrays (FPGAs), a group of processing components, or other suitable electronic processing structures. In some embodiments, processor 204 is configured to execute program code stored on memory 210 to cause Permissioning Tool 200 to perform one or more operations, as described below in greater detail. It will be appreciated that, in embodiments where Permissioning Tool 200 is part of another computing device, the components of Permissioning Tool 200 may be shared with, or the same as, the host device. For example, if Permissioning Tool 200 is implemented via a server, then Permissioning Tool 200 may utilize the processing circuit(s), processor(s), and/or memory of the server to perform the functions described herein.

Memory 210 can include one or more devices (e.g., memory units, memory devices, storage devices, etc.) for storing data and/or computer code for completing and/or facilitating the various processes described in the present disclosure. In some embodiments, memory 210 includes tangible (e.g., non-transitory), computer-readable media that stores code or instructions executable by processor 204. Tangible, computer-readable media refers to any physical media that is capable of providing data that causes Permissioning Tool 200 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 210 can include random access memory (RAM), read-only memory (ROM), hard drive storage, temporary storage, non-volatile memory, flash memory, optical memory, or any other suitable memory for storing software objects and/or computer instructions. Memory 210 can include database components, object code components, script components, or any other type of information structure for supporting the various activities and information structures described in the present disclosure. Memory 210 can be communicably connected to processor 204, such as via processing circuit 202, and can include computer code for executing (e.g., by processor 204) one or more processes described herein.

While shown as individual components, it will be appreciated that processor 204 and/or memory 210 can be implemented using a variety of different types and quantities of processors and memory. For example, processor 204 may represent a single processing device or multiple processing devices. Similarly, memory 210 may represent a single memory device or multiple memory devices. Additionally, in some embodiments, Permissioning Tool 200 may be implemented within a single computing device (e.g., one server, one housing, etc.). In other embodiments, Permissioning Tool 200 may be distributed across multiple servers or computers (e.g., that can exist in distributed locations). For example, Permissioning Tool 200 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers.

Memory 210 is shown to include a preapproval decision engine 212, which is configured to generate preapproval decisions for medical procedures and/or tests based on a patient's EMR. As shown, preapproval decision engine 212 may further include a natural language processing (NLP) model 214, a clinical appropriateness model 216, and a summary generator 218. Initially, preapproval decision engine 212 obtains a recommendation from a health care provider to conduct a procedure or test on a patient. In some implementations, the recommendation is received directly from the health care provider. For example, Permissioning Tool 200 may be directly accessible to users via a software application, a web interface, or the like, such that the recommendation may be entered into a user interface presented on provider computing system 102. In some implementations, preapproval decision engine 212 is configured to monitor multiple EMRs (e.g., all or part of EMR database 106) to detect changes (e.g., additions, updates, etc.) and, when a change is detected, to determine whether the change includes a recommendation for a procedure or test.

In some implementations, a patient's EMR is transmitted or otherwise made available to preapproval decision engine 212 during or after a visit with the health care provider. For example, once a visit with the patient is concluded, the health care provider may submit notes and other updates to the patient's EMR. In some such implementations, the patient's EMR is transmitted with the recommendation for a procedure or test to Permissioning Tool 200. In some implementations, receiving a recommendation for a procedure or test prompts preapproval decision engine 212 to retrieve the patient's EMR from a database or other system/device (e.g., EMR database 106). In some implementations, upon receiving a patient's EMR and/or notes from a health care provider relating to a visit, preapproval decision engine 212 utilizes NLP model 214 to evaluate the EMR and/or notes to determine whether the EMR and/or notes contain a recommendation for a procedure or test.

As mentioned above, for example, an EMR may contain one or more unstructured (or semi-structured) data elements, including notes (e.g., observations, recommendations, etc.) from a health care provider relating to the patient. Accordingly, NLP model 214 is generally an AI model (e.g., a machine learning model, such as a neural network) that is trained to process text to identify words, phrases, and other information relevant to determining clinical appropriateness of a medical procedure or test. In some such implementations, this identification and extraction of relevant words, phrases, and other information from text is referred to as “keyword extraction.” Generally, NLP model 214 can be or include any suitable NLP model, such as BERT (or variations thereof), XLNet, and the like. In some implementations, NLP mode 214 is trained using a training data set containing text from various EMRs, including a plurality of example notes entered by health care providers. In some implementations, NLP model 214 is configured to process various text elements (e.g., free-text) in a patient's EMR to determine whether a health care provider has recommended a procedure or test. In some implementations, NLP model 214 is used to extract data from text in the patient's EMR that is relevant to determining clinical appropriateness of a procedure or test, as mentioned above.

In addition to using NLP model 214 to extract relevant information from the unstructured data of a patient's EMR, preapproval decision engine 212 can also be configured to extract relevant information/data points from the structured data elements of the EMR. In some such implementations, preapproval decision engine 212 can determine which data points in the patient's EMR are relevant to making a preapproval decision based on the recommended medical procedure or test. For example, each different type of procedure or test may have a corresponding set of clinical appropriateness criteria that is maintained by Permissioning Tool 200. In some implementations, clinical appropriateness criteria are stored in a database 224 which may be maintained by Permissioning Tool 200, or which may be maintained/hosted remotely by another computing device. In some implementations, these clinical appropriateness criteria determine which data elements of an EMR preapproval decision engine 212 will extract and/or consider.

Generally, clinical appropriateness criteria are values, thresholds, data elements, and other parameters that an EMR must contain and/or that data elements within the EMR must meet for a medical procedure or test to be deemed clinically appropriate and/or medically necessary. In other words, clinical appropriateness criteria may be thought of as “rules” that dictate clinical appropriateness and/or medical necessity. From a determination of clinical appropriateness, preapproval decision engine 212 can make a decision as to whether a recommended procedure/test is preapproved, as discussed in greater detail below. In many cases, clinical appropriateness criteria are agreed upon by health care payors and/or providers prior to use. For example, health care payors may enter an agreement or contract with health care providers and/or an entity that operates Permissioning Tool 200 that a procedure and/or test is “clinically appropriate” and/or necessary if the patient's EMR follows a set of clinical appropriateness criteria. Thus, in some implementations, clinical appropriateness criteria are predefined. In some implementations, clinical appropriateness criteria can be added, removed, or otherwise modified by a user of Permissioning Tool 200 and/or by health care payors.

In some implementations, a unique set of clinical appropriateness criteria is established for each different type of medical procedure/test—or at least for a set of common procedures/tests—that can be recommended by a health care provider. For example, a vast number of different and/or common medical procedures/tests are generally known or at least predetermined, which can be used to establish clinical appropriateness criteria. Thus, database 224 may contain a plurality of different sets of clinical appropriateness criteria associated with each of a plurality of different procedures/tests. When a procedure/test is recommended by a health care provider, the associated set of clinical appropriateness criteria may then be referenced when determining clinical appropriateness. Accordingly, in some implementations, preapproval decision engine 212 is configured to identify an appropriate set of clinical appropriateness criteria for a recommended procedure/test.

Using the unstructured data extracted by NLP model 214 and/or other structured data extracted by preapproval decision engine 212, from a patient's EMR, clinical appropriateness model 216 can generate a clinical appropriateness decision and/or score. Specifically, in some implementations, the extracted data from the patient's EMR are provided as inputs to clinical appropriateness model 216, which outputs a determination or prediction as to whether the recommended medical procedure/test is “clinically appropriate.” In some implementations, clinical appropriateness model 216 is a rules-based model, which maps or compares the extracted data to a set of clinical appropriateness criteria associated with the recommended procedure/test. In some such implementations, the rules-based model outputs a binary results (e.g., a “yes”/“no” or “clinically appropriate”/“not clinically appropriate”) or a score. A binary result may be established if the data from the patient's EMR matches or meets at least a certain subset of the clinical appropriateness criteria for the procedure/test. A score may be generated based on the number or amount of data elements that match or meet the clinical appropriateness criteria. In some implementations, a score may be used to generate a binary result if the score meets or exceeds a threshold value.

In some implementations, clinical appropriateness model 216 is a classification model. More specifically, in some such implementations, clinical appropriateness model 216 is a machine learning model (e.g., a neural network) or other AI model that identifies a class based on input data. In some such implementations, clinical appropriateness model 216 includes one or more multi-layer perceptrons (MLPs), support vector machines (SVMs), random forest models, or convolutional neural networks (CNNs); although it should be appreciated that the present description is not limited to only these types of classification models. In some implementations, the classification model is a type of artificial neural network (ANN). A CNN is a type of deep neural network that has been applied, for example, for classification of input data. Unlike a traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks. An SVM is a supervised learning model that uses statistical learning frameworks to predict the probability of a target. This disclosure contemplates that the SVMs can be implemented using a computing device (e.g., a processing unit and memory as described herein). SVMs can be used for classification and regression tasks. SVMs are trained with a data set (also referred to herein as a “dataset”) to maximize or minimize an objective function, for example a measure of the SVM's performance, during training. An ANN having hidden layers can also be referred to as deep neural network or MLP.

In some implementations, clinical appropriateness model 216 includes multiple different classification models each trained to determine clinical appropriateness for a different procedure/test. For example, after preapproval decision engine 212 determines the procedure or test recommended by a health care provider, clinical appropriateness model 216 may select an appropriate classification model for determining clinical appropriateness. In some implementations, clinical appropriateness model 216 provides the extracted data from the patient's EMR to a suitable one of the classification models to determine a classification of either “yes”/“no” or “clinically appropriate”/“not clinically appropriate”. In other implementations, clinical appropriateness model 216 includes a single classification model that attempts to classify the input data (e.g., the extracted data from the patient's EMR) as clinically appropriate for a variety of different procedures/tests. Put another way, clinical appropriateness model 216 may determine whether the extract data indicates clinical appropriateness for multiple different procedures and tests. If the model cannot find a suitable classification based on the EMR data, then a “not clinically appropriate” result may be returned.

In some implementations, clinical appropriateness model 216 and/or the machine learning models implemented by clinical appropriateness model 216 are trained using a training data set built from one or more of: i) medical guidelines, such as those provided by regulatory body (e.g., Centers for Medicare & Medicaid Services (CMS)); ii) historical care data, including historical care recommendations and acceptance/approvals from health care payors; and iii) historical health care claims or claims derivative data, including health care claims submitted post-encounter (e.g., post visit between a health care provider and a patient) to a health care payor, which can be tied back to earlier/previously recommended care. In some implementations, medical guidelines can be obtained from one or more remote systems or databases. For example, medical guideline data can be requested from various regulatory bodies and/or retrieved from web sites or other computing systems associated with the various regulatory bodies. In some implementations, historical care data can include “prior-authorization-obtained” pre-care/at-care/or post-care. It should be appreciated that the various data used to construct a training data set for training clinical appropriateness model 216 and/or the machine learning models thereof can be continuously or periodically updated (e.g., as new data is added). For this reason, in some implementations, clinical appropriateness model 216 and/or the machine learning models thereof may be periodically retrained based on new and/or updated training data, to provide more accurate predictions and/or determinations of clinical appropriateness.

In implementations where clinical appropriateness model 216 includes one or more machine learning models (e.g., classification models), each machine learning model may be trained on an appropriate data set to determine a class label or classification for input data. In some such implementations, multiple classification models can be trained, each to determine clinical appropriateness of a specific procedure/test. In other such implementations, as mentioned above, a single model or a select few models can be trained to determine whether input data is “clinically appropriate” for any of a number of different procedures/tests. In either case, the classification models are generally trained using suitable learning techniques, such as supervised learning, in order to accurately classify the input data.

In some implementations, clinical appropriateness model 216 generates a score indicative of clinical appropriateness. As mentioned above, the score may be determined using a rules-based model. In some such implementations, the score may be calculated based on a number or amount of clinical appropriateness criteria that the patient's EMR meets or matches. In other implementations, where clinical appropriateness model 216 uses a classification model to predict clinical appropriateness, the score can be a confidence value or probability that the input data (e.g., the patient's EMR data) can be classified as “clinically appropriate” for the procedure/test. In some implementations, the score is a value between ‘O’ and ‘l’ or a percentage. For example, a score of 0.7 may indicate a 70% confidence in the classification or a 70% probability that that input data meets the classification criteria. In some implementations, if the clinical appropriateness score (e.g., the probability output by a classification model or a score generated by a rules-based model) meets or exceeds a threshold, then preapproval decision engine 212 determines that the procedure/test is preapproved; otherwise, the procedure/test may be denied. In some implementations, clinical appropriateness model 216 simply outputs a preapproval decision or classification without separately generating a score.

Consider, for example, a recommendation from a health care provider that a patient with burns undergo a skin graft procedure. Upon obtaining the recommendation, preapproval decision engine 212 may extract relevant data from the patient's EMR (e.g., using NLP model 214 for the unstructured and/or text-based data) and may provide the extracted data as an input to clinical appropriateness model 216 in order to generate a clinical appropriateness score/decision. Relevant data may include, for example, a severity of the burns, an indication of how much of the patient's body is covered in burns, patient biometric and demographic information, and the like. In implementations where clinical appropriateness model 216 is or includes rules-based models, clinical appropriateness model 216 may map the extracted data to clinical appropriateness criteria to generate the score/decision. In implementations where clinical appropriateness model 216 is or includes classification models, clinical appropriateness model 216 may execute an appropriate classification model using the extracted data to determine a classification and/or probability score. Preapproval decision engine 212 may then compare this score to a threshold (e.g., 0.7) to determine preapproval. Say, in this example, that clinical appropriateness model 216 returns a score of 0.8 and that the threshold is 0.7; accordingly, the recommendation for a skin graft may be preapproved as the clinical appropriateness score exceeds the threshold.

Once determined, an indication of the preapproval decision may be returned to the health care provider (e.g., via provider computing system 102). In some implementations, the indication of the preapproval decision (e.g., approval or denial) is displayed on a user interface of provider computing system 102 or another computing device operated by the health care provider. In addition, summary generator 218 can be configured to generate a summary of how the preapproval decision was determined. More specifically, summary generator 218 can generate a summary that indicates one or more criteria that were considered and/or that impacted the clinical appropriateness score. In the preceding example of a patient with burns, the summary may indicate the various clinical appropriateness criteria that were considered (e.g., burn severity, burn coverage, etc.) and whether the patient's EMR met each criteria. In addition, the summary may include an indication of the preapproval decision. Optionally, the summary can also include all or part of the patient's EMR. For example, the summary may include the data that was extracted from the patient's EMR and/or the notes from the health care provider that prompted the clinical appropriateness assessment. The generated summary may then be transmitted to one or both of the health care provider and a payor (e.g., payor computing system 104).

In some implementations, memory 210 further includes a claims generator 220 configured to auto-generate electronic health care claims. In particular, claims generator 220 may generate electronic health care claims for procedures/tests that are preapproved by preapproval decision engine 212. For example, once preapproval decision engine 212 generates a preapproval decision, claims generator 220 can generate a relevant electronic health care claim directed to the procedure/test that was being evaluated for clinical appropriateness. As described herein, an electronic health care claim generally refers to a health care claim that is electronically generated and/or submitted for adjudication and payment. In some implementations, the electronic health care claim is formatted as an EDI 837 “Healthcare Claim,” as understood by those of ordinary skill in the art. In some implementations, claims generator 220 is configured to automatically identify and/or determine claim codes-including diagnosis, procedure, and/or other billing codes—by executing an NLP on the health care provider's notes included in the patient's EMR. In some implementations, the NLP used for claim coding is the same as NLP model 214 or a different NLP. For example, the NLP implemented by claims generator 220 may be BERT or a variation thereof, XLNet, or any other suitable model. Once the diagnosis, procedure, and/or other billing codes are identified, claims generator 220 may generate and/or format the electronic health care claim. In some implementations, the auto-generated electronic health care claim is sent to one or both of the health care provider and payor (e.g., provider computing system 102 and/or payor computing system 104). In some implementations, the auto-generated electronic health care claim is part of a consolidated data package sent to the health care payor, which includes the summary generated by summary generator 218.

Optionally, memory 210 can include a settlement engine 222 configured to settle electronic health care claims; particularly, the electronic health care claims that are auto-generated by claims generator 220. In some implementations, settlement engine 222 can communicate with a health care payor (e.g., payor computing system 104) facilitate adjudication of electronic health care claims and/or to receive payment for electronic health care claims. For example, upon adjudicating the received electronic health care claim (generated by claims generator 220), payor computing system 104 may transmit payment for the electronic health care claim to settlement engine 222 for preliminary settlement. In some implementations, settlement engine 222 holds the payment, such as until the associated procedure/test is complete. In some implementations, settlement engine 222 facilitates the deposit of funds into a bank account or fund associated with the health care payor.

Still referring to FIG. 2, Permissioning Tool 200 is also shown to include a communications interface 230 that facilitates communications between Permissioning Tool 200 and any external components or devices, including provider computing system 102 and payor computing system 104. Accordingly, communications interface 230 can be, or can include, any number and/or combination of wired or wireless communications interfaces (e.g., jacks, antennas, transmitters, receivers, transceivers, wire terminals, etc.) for conducting data communications. In some embodiments, communications via communications interface 230 are direct (e.g., local wired or wireless communications) or via a network (e.g., a WAN, the Internet, a cellular network, etc.). For example, communications interface 230 may include one or more Ethernet ports for communicably coupling Permissioning Tool 200 to a network (e.g., the Internet). In another example, communications interface 230 can include a Wi-Fi transceiver for communicating via a wireless communications network. In yet another example, communications interface 230 may include cellular or mobile phone communications transceivers.

In some implementations, communications to and/or from Permissioning Tool 200 may be facilitated by a suitable application programming interface (API). An API is generally an interface that allows remote and/or third-party computing systems to send data to or request data from Permissioning Tool 200 and may be any suitable API, such as a RESTful API.

Accordingly, in some implementations, Permissioning Tool 200 may be cloud-based (e.g., hosted on a cloud server) and an API may facilitate communications with provider computing system 102 and/or payor computing system 104. Additionally, in some implementations, Permissioning Tool 200 may be accessible from a user device (e.g., a personal computer) such that a user can view or change data or otherwise control Permissioning Tool 200. In some implementations, the user device is any computing device that is not a provider or payor computing device. For example, the user device may be a computer operated by a user that manages Permissioning Tool 200.

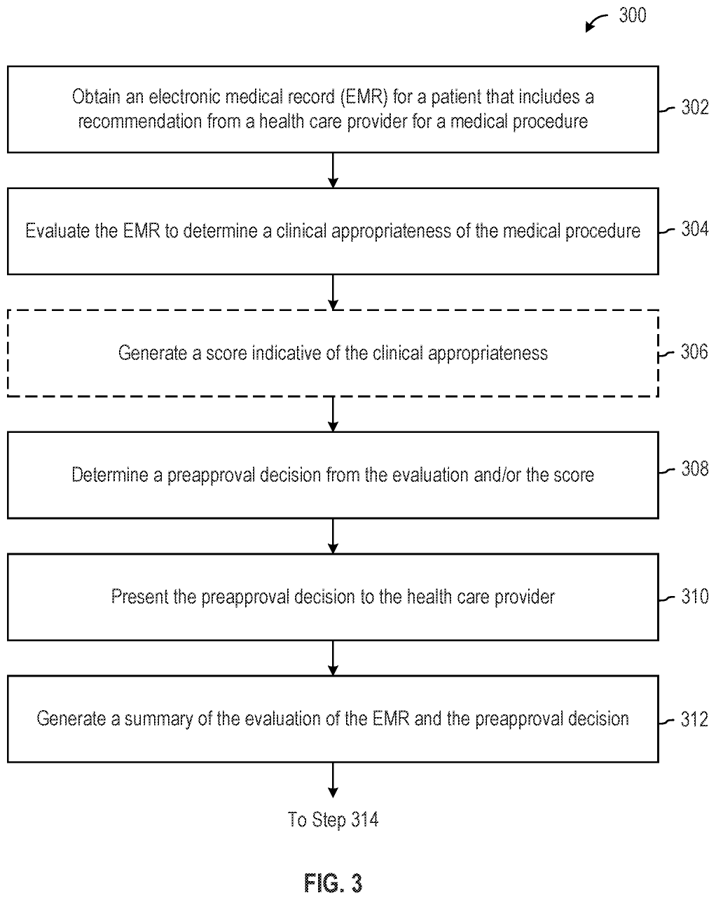

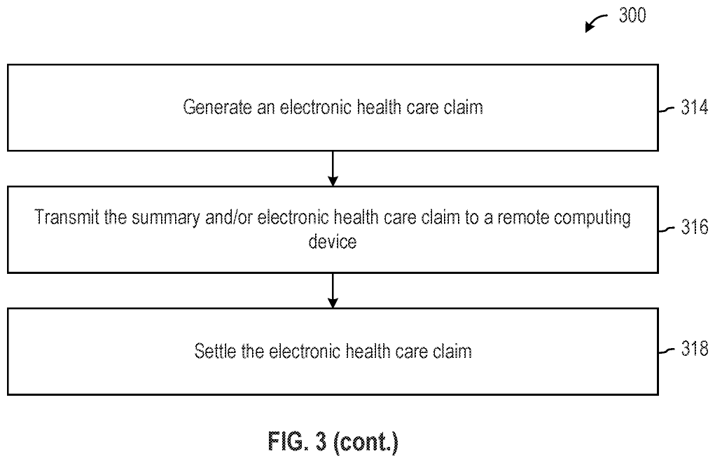

Referring now to FIG. 3, a flow chart of a process 300 for automated preapproval of medical procedures and tests is shown, according to some implementations. Notably, as discussed above, process 300 can allow health care professionals to proceed with performing procedures and tests without seeking prior approval from payors, which decreases wait times and reduces the burden on health care payors in reviewing requests for preapproval. Further, process 300 can reduce health care claims adjudication and settlement times. In some implementations, process 300 is at least partially implemented or executed by Permissioning Tool 200. It will also be appreciated that certain steps of process 300 may be optional and, in some implementations, process 300 may be implemented using less than all of the steps. It will also be appreciated that the order of steps shown in FIG. 3 is not intended to be limiting.

At step 302, an EMR for a patient, which includes a recommendation from a health care provider for a medical procedure/test, is obtained. In some implementations, the EMR is obtained directly from a health care provider. For example, the health care provider may transmit the EMR directly to Permissioning Tool 200. As another example, the health care provider may enter EMR data (e.g., patient information, such as biometrics, notes, etc.) into a web interface, a software application, or other user interface for Permissioning Tool 200. In some implementations, the EMR is obtained indirectly, such as by retrieving the EMR from a database or other computing system. In some implementations, a patient's EMR is obtained during or after a visit with the health care provider. For example, once a visit with the patient is concluded, the health care provider may submit notes and other updates to the patient's EMR. In some implementations, the patient's EMR (e.g., as part of a database of EMRs) may be monitored to automatically detect changes (e.g., additions, updates, etc.). In some implementations, the EMR is evaluated to determine whether the EMR and/or notes contain a recommendation for a procedure or test. For example, a patient's EMR may be automatically evaluated for recommended procedures/tests if changes are detected and/or as part of a periodic scan.

As mentioned above, the EMR generally includes both structured data (e.g., biometric data, such as height, weight, heart rate, blood pressure, etc.) and unstructured data, wherein the unstructured data includes notes or other text-based entries provided by the health care provider. For example, the EMR may include notes on the patient's condition, answers to diagnostic questions, and the like. In addition, the EMR may contain at least a portion of the patient's medical history. In some cases, the EMR will include (e.g., in the notes) a recommendation from the health care provider to perform or conduct a medical procedure or diagnostic test on the patient. In other cases, the health care provider may request the medical procedure or diagnostic test separately from the patient's EMR. For example, the health care provider may submit a request for preapproval of the recommended procedure/test separate from the patient's EMR. In other words, rather than receiving a patient's EMR directly, a request for preapproval of a procedure/test is received. In some such implementations, upon receiving the request, Permissioning Tool 200 may retrieve the patient's EMR for further evaluation.

At step 304, the EMR is evaluated to determine the clinical appropriateness of the medical procedure/test. As discussed above, clinical appropriateness may be determined based on predefined criteria or parameters, which are typically agreed to by health care payors and/or providers. In some cases, different types of medical procedures and tests can have different clinical appropriateness criteria. To determine clinical appropriateness of the medical procedure/test, at least a subset of both the structured and unstructured data can be extracted from the electronic medical record. The extracted data is generally data that is relevant to the medical procedure or test. In some implementations, the extracted data is identified based on the clinical appropriateness criteria. For example, the clinical appropriateness criteria may define a set of data points that are desirable or necessary for making a clinical appropriateness determination. In this regard, clinical appropriateness criteria associated with the procedure/test that is being evaluated for preapproval may first be identified and then referenced for data extraction.

In some implementations, an NLP model or other AI model is used to extract relevant data elements from one or both of the structured and unstructured data in the patient's EMR. For example, an NLP model can be used on at least any text in the unstructured data elements of the EMR to identify/extract various data points. In some such implementations, an NLP model is used to perform keyword extraction on text in the EMR, which is used to determine clinical appropriateness. For example, answers to diagnostic questions as provided by a patient may be extracted from the health care provider's notes, as they may be relevant to determining clinical appropriateness.

Once the data relevant to the medical procedure/test is extracted from the patient's EMR, it may be provided as an input to a “clinical appropriateness” model (e.g., clinical appropriateness model 216). As discussed above, a “clinical appropriateness” model is generally configured to output either a decision/prediction of whether the medical procedure/test is clinically appropriate or a score indicative of clinical appropriateness, based on the patient's EMR. In other words, the data points extracted from the EMR are provided as inputs to the model, and the model outputs either a clinical appropriateness decision or score. In implementations where the model outputs a score, process 300 may continue to step 306, below. Otherwise, if the model outputs a clinical appropriateness decision/prediction, process 300 may continue to step 308.

In some implementations, the model is a rules-based model which maps or otherwise compares the extracted data to predefined clinical appropriateness criteria. In other words, the rules-based model compares data points extracted from both the structured and unstructured data of the EMR to predefined criteria to determine whether each of the criteria are satisfied. As mentioned above, clinical appropriateness criteria may vary for different procedures/tests. For example, the criteria used to determine the appropriateness of a CT scan for headaches may be different from the criteria used to determine the appropriateness of a knee surgery. Accordingly, in some implementations, step 304 may include selecting/identifying an appropriate set of criteria based on the medical procedure/test being evaluated.

In some implementations, the model is a classification model which predicts a class label or classification (e.g., “clinically appropriate”/“not clinically appropriate”) based on the extracted data. Additionally, or alternatively, the classification model may output a probability that input data fits one or more classes. In some such implementations, the classification model is a machine learning model, as described above. For example, the classification model may be an MLP, an SVM, or the like. In some implementations, and again because clinical appropriateness criteria may vary for different procedures/tests, multiple classification models may be generated, trained, and/or otherwise made available to make clinical appropriateness decisions for different medical procedures/tests. In some such implementations, step 304 may include selecting an appropriate model from a set of available models. For example, if the procedure/test is an MRI scan due to a head injury, a suitably trained classification model may be selected/identified. Alternatively, in some implementations, a single classification model can be trained to classify input data as either “not clinically appropriate” or to identify one or more medical procedures/tests that would be clinically appropriate. In other words, the classification model can identify one or more medical procedures/tests that would be clinically appropriate based on the patient's EMR, in which case Permissioning Tool 200 can determine whether the requested/recommended procedure/test matches any of the clinically appropriate procedures/tests.

At step 306, a score indicative of clinical appropriateness is generated. Generally, this score is output by the clinical appropriateness model at step 304. In some implementations, a rules-based model outputs a score that is calculated based on the number or amount of data elements extracted from the patient's EMR that match or meet the clinical appropriateness criteria. In some implementations, a classification model outputs a confidence or probability score. A confidence or probability score generally indicates a confidence/probability in a predicted class for the input data. In some implementations, the score is a value between ‘0’ and ‘1’ or a percentage. For example, a score of 0.7 may indicate a 70% confidence in the classification or a 70% probability that that input data meets the classification criteria. In some implementations, step 306 is optional, as the clinical appropriateness model(s) may be configured to directly output a classification/clinical appropriateness decision.

At step 308, a preapproval decision is determined based on the clinical appropriateness evaluation/score. In some implementations, the medical procedure/test is preapproved if the clinical appropriateness model determines that the medical procedure/test is clinically appropriate. In some implementations, the clinical appropriateness model itself is configured to output a determination of “approved”/“denied.” In some implementations, where the clinical appropriateness model generates a clinical appropriateness score, the score may be compared to a threshold value, where, if the score meets or exceeds the threshold, the procedure/test is considered to be preapproved. For example, if the model outputs a score of 0.8 and the threshold is 0.5, the procedure/test is preapproved.

At step 310, the preapproval decision is presented to the health care provider that submitted the recommendation/request. In particular, the preapproval decision may be transmitted to the health care provider's computing device (e.g., provider computing system 102) for display via a user interface. Additionally, or alternatively, the preapproval decision may be transmitted to another remote device. In some implementations, receiving the preapproval decision causes a computing device to display the decision.

At step 312, a summary of the preapproval decision is generated. Generally, the summary includes an indication of the preapproval decision and an indication of clinical appropriateness criteria that impacted the preapproval decision. In other words, the summary may indicate one or more criteria that were considered and/or that impacted the clinical appropriateness evaluation and/or that affected the score. For example, the summary may indicate data from the patient's EMR that was influential in determining the clinical appropriateness, which criteria were considered, how each data element from the patient' EMR compared to the criteria, and the like. Optionally, the summary can also include all or part of the patient's EMR. For example, the summary may include the data that was extracted from the patient's EMR and/or the notes from the health care provider that prompted the clinical appropriateness assessment.

At step 314, an electronic health care claim is generated for the medical procedure/test. As discussed above, the electronic health care claim may be generated by automatically identifying diagnosis, procedure, and/or other billing codes from the patient's EMR. In some implementations, the electronic health care claim is generated only if the procedure/test is preapproved. In some implementations, diagnosis, procedure, and/or other billing codes are automatically identified using an NLP model, with the health care provider's notes included in the patient's EMR being the input to the NLP model.

At step 316, the summary and/or the electronic health care claim is transmitted to a remote computing device. In many cases, the remote computing device is associated with the health care payor (e.g., payor computing system 104). In some implementations, only the summary is transmitted to the remote computing device. In some implementations, both the summary and the patient's EMR, or at least a portion of the patient's EMR, are transmitted to the remote device in a consolidated data package. In some implementations, the consolidated data package further includes the auto-generated electronic health care claim.

At step 318, the electronic health care claim is (optionally) preliminarily settled with the health care payor. In some implementations, the health care payor (e.g., payor computing system 104) adjudicates the electronic health care claims, such as by preforming a spot adjudication/auditing, reviewing the summary for accuracy, and the like. Once the payor is satisfied with the claim, the payor may submit payment to Permissioning Tool 200 for preliminary settlement. In some implementations, Permissioning Tool 200 holds the payment, such as until the associated procedure/test is complete. In some implementations, Permissioning Tool 200 facilitates the deposit of funds into a bank account or fund associated with the health care payor.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A system comprising:
a processor; and
memory having instructions stored thereon that, when executed by the processor, cause the system to:
obtain an electronic medical record for a patient, wherein the electronic medical record includes a recommendation from a health care provider for a medical procedure or test and a medical history for the patient stored as both structured and unstructured data;
determine a clinical appropriateness of the medical procedure or test by: i) extracting a subset of both the structured and unstructured data from the electronic medical record relevant to the medical procedure or test, and ii) generating a score indicative of the clinical appropriateness based on the subset of both the structured and unstructured data;
generate a preapproval decision for the medical procedure or test based on the score, wherein the medical procedure or test is preapproved if the score meets or exceeds a threshold value;
present the preapproval decision to the health care provider;
generate a summary of the preapproval decision, wherein the summary includes an indication of the preapproval decision and an indication of clinical appropriateness criteria that impacted the preapproval decision; and
transmit the summary to a remote computing device.

2. The system of claim 1, wherein the unstructured data included in the electronic medical record includes text-based notes entered by the health care provider, and wherein extracting the subset of both the structured and unstructured data includes performing keyword extraction on the text-based notes using a natural language processing model.

3. The system of claim 1, wherein the score indicative of the clinical appropriateness is generated using a classification model, wherein the classification model is a machine learning model, and wherein the subset of both the structured and unstructured data are provided as inputs to the classification model.

4. The system of claim 1, wherein the score indicative of the clinical appropriateness is generated using a rules-based model, wherein the rules-based model compares data points of the subset of both the structured and unstructured data to a set of clinical appropriateness criteria.

5. The system of claim 1, wherein the instructions further cause the system to:
identify procedure and diagnostic codes from the electronic medical record using an artificial intelligence model;
generate an electronic health care claim based on the procedure and diagnostic codes; and
transmit the electronic health care claim to the remote computing device.

6. The system of claim 5, wherein the instructions further cause the system to:
receive payment for the electronic health care claim from the remote computing device; and
process the payment to preliminarily settle the electronic health care claim.

7. The system of claim 1, wherein to obtain the electronic medical record for the patient, the instructions further cause the system to:
receive a request for preapproval of the medical procedure or test from the health care provider; and
retrieve the electronic medical record from a database.

8. The system of claim 1, wherein presenting the preapproval decision to the health care provider comprises at least one of: i) transmitting the preapproval decision to a second remote computing device, or ii) displaying the preapproval decision on a user interface.

9. The system of claim 1, wherein the remote computing device is a first computer associated with a health care payor, and wherein the electronic medical record is received from a second computer associated with the health care provider.

10. A computer-implemented method comprising:
obtaining an electronic medical record for a patient, wherein the electronic medical record includes a recommendation from a health care provider for a medical procedure or test and a medical history for the patient stored as both structured and unstructured data;
determining a clinical appropriateness of the medical procedure or test by: i) extracting a subset of both the structured and unstructured data from the electronic medical record relevant to the medical procedure or test, and ii) generating a score indicative of the clinical appropriateness based on the subset of both the structured and unstructured data;
generating a preapproval decision for the medical procedure or test based on the score, wherein the medical procedure or test is preapproved if the score meets or exceeds a threshold value;
presenting the preapproval decision to the health care provider;
generating a summary of the preapproval decision, wherein the summary includes an indication of the preapproval decision and an indication of clinical appropriateness criteria that impacted the preapproval decision; and
transmitting the summary to a remote computing device.

11. The computer-implemented method of claim 10, wherein the unstructured data included in the electronic medical record includes text-based notes entered by the health care provider, and wherein extracting the subset of both the structured and unstructured data includes performing keyword extraction on the text-based notes using a natural language processing model.

12. The computer-implemented method of claim 10, wherein the score indicative of the clinical appropriateness is generated using a classification model, wherein the classification model is a machine learning model, and wherein the subset of both the structured and unstructured data are provided as inputs to the classification model.

13. The computer-implemented method of claim 10, wherein the score indicative of the clinical appropriateness is generated using a rules-based model, wherein the rules-based model compares data points of the subset of both the structured and unstructured data to a set of clinical appropriateness criteria.

14. The computer-implemented method of claim 10, further comprising:
identifying procedure and diagnostic codes from the electronic medical record using an artificial intelligence model;
generating an electronic health care claim based on the procedure and diagnostic codes; and
transmitting the electronic health care claim to the remote computing device.

15. The computer-implemented method of claim 14, further comprising:
receiving payment for the electronic health care claim from the remote computing device; and
processing the payment to preliminarily settle the electronic health care claim.

16. The computer-implemented method of claim 10, wherein obtaining the electronic medical record for the patient includes:
receiving a request for preapproval of the medical procedure or test from the health care provider; and
retrieving the electronic medical record from a database.

17. The computer-implemented method of claim 10, wherein presenting the preapproval decision to the health care provider comprises at least one of: i) transmitting the preapproval decision to a second remote computing device, or ii) displaying the preapproval decision on a user interface.

18. The computer-implemented method of claim 10, wherein the summary further includes a copy of the electronic medical record or a portion of the electronic medical record.

19. The computer-implemented method of claim 10, wherein the electronic medical record is obtained upon detecting that the electronic medical record has been created or updated by the health care provider.

20. A non-transitory computer readable medium having instructions stored thereon that, when executed by a processor, cause a device to:
obtain an electronic medical record for a patient, wherein the electronic medical record includes a recommendation from a health care provider for a medical procedure or test and a medical history for the patient stored as both structured and unstructured data;
determine a clinical appropriateness of the medical procedure or test by: i) extracting a subset of both the structured and unstructured data from the electronic medical record relevant to the medical procedure or test, and ii) generating a score indicative of the clinical appropriateness based on the subset of both the structured and unstructured data;
generate a preapproval decision for the medical procedure or test based on the score, wherein the medical procedure or test is preapproved if the score meets or exceeds a threshold value;
present the preapproval decision to the health care provider;
generate a summary of the preapproval decision, wherein the summary includes an indication of the preapproval decision and an indication of clinical appropriateness criteria that impacted the preapproval decision; and
transmit the summary to a remote computing device.
